(19) United States
(12) Patent Application Publication
(10) Pub. No.: US 2001/0031783 A1
(43) Pub. Date: Oct. 18, 2001

(54) Alpha-pyrone compositions and method for the chemoprevention of cancer

(76) Inventor: Gregory Gene Steiner, Honolulu, HI (US)

Correspondence Address: Gregory Gene Steiner, P O Box 61515, Honolulu, HI, 96839, US

(21) Appl. No.: 09/792,898

(22) Filed: Feb. 26, 2001

Related U.S. Application Data
(60) Provisional application No. 60/186,688, filed on Mar. 3, 2000.

(51) Int. Cl. A61K 31/351

(52) U.S. Cl. 514/460

(57) ABSTRACT

A method of chemopreventing cancer by administering chemopreventive compositions of matter in an effective amount of at least one composition from a specific group of alpha-pyrones detailed in this invention formulated into a physiologically acceptable carrier medium.

### BACKGROUND-FIELD OF THE INVENTION

The present invention relates to a novel use of therapeutic compositions comprising at least one alpha-pyrone as the active principal thereof, and to the use of such novel compositions for the prevention of cancer. In particular, this invention relates to cancer chemoprevention in mammals, including humans, utilizing a specific group of alpha-pyrones as cancer chemopreventive agents.

### BACKGROUND-DESCRIPTION OF PRIOR ART

Cancer prevention is now a well established medical science. Chemoprevention has been described as the intervention with specific agents to prevent, inhibit or reverse carcinogenesis before malignancy. At this time there is a concerted effort to find effective chemopreventive agents for cancer and also to subject these agents to mechanistic studies to determine their mode of action.

It is estimated that the human diet plays a causative role in over one-third of human cancer. However, the diet does not only contains carcinogens, but also contains a variety of compounds that blocks carcinogenesis. Therefore chemoprevention is the preferable way to reduce cancer mortality and morbidity. The alpha-pyrone compounds described in this invention have demonstrated a very strong inverse correlation when comparing the consumption of the alpha-pyrones described and the incidence of cancer. In populations that consume the alpha-pyrones described in this invention there is a direct relationship between the amount of alpha-pyrones consumed and a reduction in the incidence rate of cancer (Steiner 2000).

Patents have been issued that involve the use of alpha-pyrones in mammals. U.S. Pat. No. 5,585,386 cites the use of the same group of alpha-pyrones detailed in this invention for the use of stimulating hair growth. U.S. Pat. No. 5,981,496 describes a new group of alpha-pyrones for the treatment of cancer. In this patent the group of alpha-pyrones is different for the alpha-pyrones detailed in this invention. Also the alpha-pyrones described in U.S. Pat. No. 5,981,496 are used to treat cancer after it has already become established. The alpha-pyrones detailed in this invention are used for the prevention of cancer.

### SUMMARY OF THE INVENTION

The object of the present invention is the provision of compounds of a specific alpha-pyrone type for the prevention of cancer.

Briefly, the present invention features novel therapeutic compositions for the prevention of cancer comprising in a physiologically acceptable medium, at least one alpha-pyrone having the following structural formula:

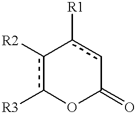

in which R1 is a hydrogen atom or an alkoxy radical having 1 to 4 carbon atoms, R2 is a hydrogen atom or a hydroxyl group, and R3 is an alkyl radical having from 1 to 4 carbon atoms or a styryl or phenethyl radical optionally substituted by one or two methylenedioxy radicals or one or two hydroxyl groups and/or one or two alkoxy radicals having from 1 to 4 carbon atoms, with the proviso that, when R2 is a hydroxyl group, then R3 is necessarily an unsubstituted phenethyl radical, with the proviso that when R3 is an alkyl radical having 1 to 4 carbon atoms, then R1 and R2 cannot both be hydrogen.

Alpha-pyrones fitting the above formula are found in the plant Piper methysticum commonly called kava. The group of alpha-pyrones found in Piper methysticum are referred to a kavapyrones.

An important aspect of the present invention is to provide a method and composition to prevent the development of cancer using the group of alpha-pyrones as described above.

Another aspect of the invention is to provide the identified group of alpha-pyrones as a food, beverage or food supplement for the prevention of cancer.

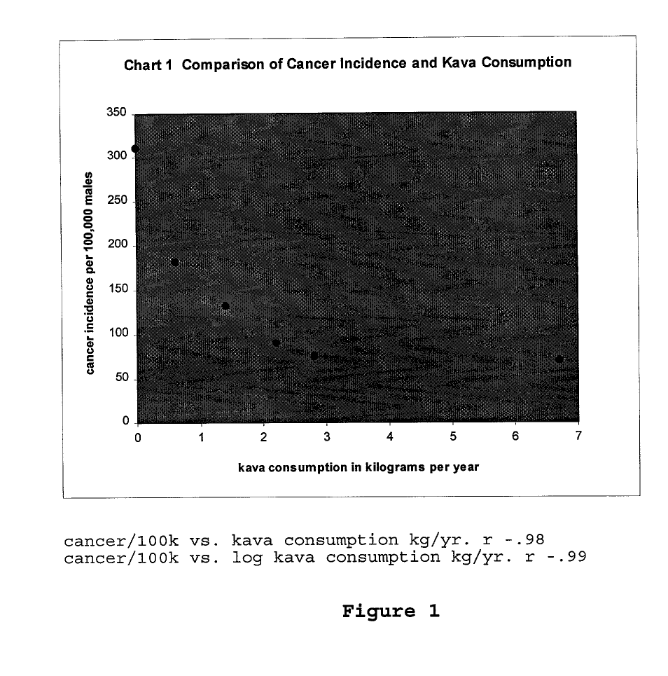

### BRIEF DESCRIPTION OF FIG. 1

FIG. 1 contains a chart of the relationship between the incidence of cancer and the amount of kava consumed by a population.

### DETAILED DESCRIPTION OF THE INVENTION

Representatives of the group of alpha-pyrones identified in this invention are naturally found in the kava plant (Piper methysticum). This invention involves the use of a group of alpha-pyrones commonly known as kavapyrones which are found in the kava plant (Piper methysticum).

The present invention features a method of chemopreventing cancer comprising in a physiologically acceptable medium, at least one alpha-pyrone having the following structural formula:

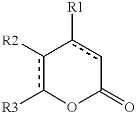

in which R1 is a hydrogen atom or an alkoxy radical having 1 to 4 carbon atoms, R2 is a hydrogen atom or a hydroxyl group, and R3 is an alkyl radical having from 1 to 4 carbon atoms or a styryl or phenethyl radical optionally substituted by one or two methylenedioxy radicals or one or two hydroxyl groups and/or one or two alkoxy radicals having from 1 to 4 carbon atoms, with the proviso that, when R2 is a hydroxyl group, then R3 is necessarily an unsubstituted phenethyl radical, with the proviso that when R3 is an alkyl radical having 1 to 4 carbon atoms, then R1 and R2 cannot both be hydrogen.

Among the alpha-pyrone compounds comprising the therapeutic compositions of the invention are the following:

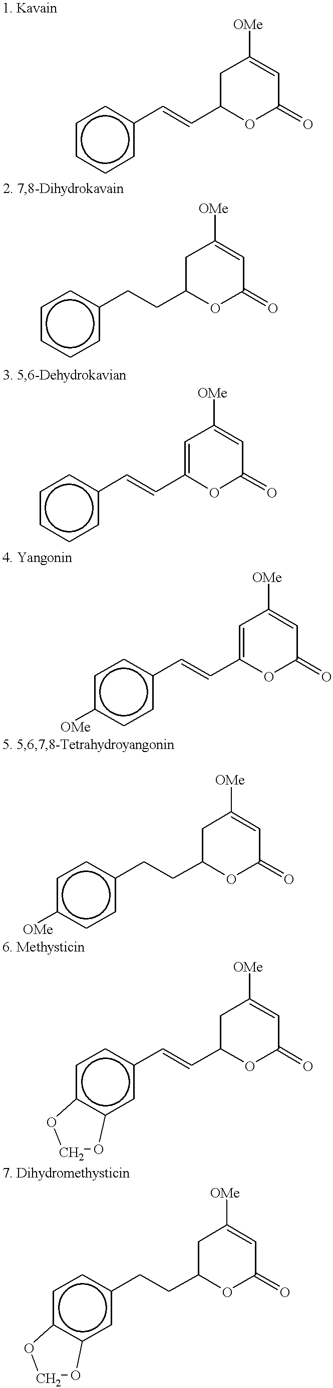

All of these alpha-pyrones compounds are per se known to this art.

The commonly accepted actions of the alpha-pyrones found in kava which are referenced in the literature are as an anti-anxiety agent (Voltz 1997), antidepressant (Warnecke G et al 1991), euphoriant (Baum S S et al., 1998), muscle relaxant (Seitz 1997), analgesic (Jamieson 1990), anticonvulsant (Kretzschmar R 1969) and as a topical treatment for hair loss (U.S. Pat. No. 5585386). Kavapyrones have become popular in the west as anti-anxiety agents. No side effects have been identified when used on a daily basis in moderate amounts (German Commission E). Years of daily use have been found to cause a dermatologic scaling which is reversed when the drug is discontinued (Norton SA et al., 1994). No irreversible side effects have been noted.

Kava is consumed much like alcohol is consumed in the west. Men will often stop at the kava bar after work and enjoy bowls of kava with friends. While women often drink kava the majority of kava consumption is by men. The most accurate numbers for the cancer rates and amount of kava consumption is from data gathered in the 1980's. Therefore the population figures that were used to calculate the kava consumption per person is based on figures form 1989.

The South Pacific Commission Cancer Registry was established in 1977. As a result of the registry many South Pacific Nations have been shown to have significantly lower cancer incidence than the other parts of the world. Table 1 lists the age-standardized cancer incidence for male and females throughout the Pacific with Los Angeles Caucasians as a reference.

TABLE 1
Age-standardized cancer incidence rates for all sites
males and females per 100,000 population

| Country | Incidence male | Incidence female |
|---|---|---|
| Vanuatu | 70.9 | 83.7 |
| (1980-1986) | | |
| Fiji | 75 | 112.2 |
| (1979-1982) | | |
| Western Samoa | 90.2 | 93.7 |
| (1980-1988) | | |
| Micronesia | 132.9 | 97.0 |
| (1980-1982) | | |
| New Caledonia | 182.0 | 154.0 |
| (1977-1981) | | |
| Hawaii/Hawaiians | 311.9* | 297.6* |
| (1978-1982) | | |
| NewZealand/Maoris | 322.9* | 297.6* |
| (1978-1982) | | |
| USA, Los Angeles | 307.2* | 276.2* |
| (1978-1982) | | |

*all sites but 173 (other skin)

Table 2 lists the cancer incidence and kilograms of kava consumed in each country. In every country the more kava consumed the lower the cancer incidence. The data from table 2 is displayed in FIG. 1. There is an inverse relationship between the cancer incidence rate and kava consumption.

TABLE 2
Country, population, kilograms consumed, cancer
incidence rate and kilograms of kava consumed per person.

| | | | Cancer | |
|---|---|---|---|---|
| | | | rate/ | |
| | Popula- | Kilograms | 100,000 | Kilograms/ |
| Country | tion 1989 | consumed | males | person |
| Vanuatu | 155,000 | 1,042,252 | 70.9 | 6.7 |
| Tonga | 100,000 | 400,000 | unknown | 4.0 |
| Fiji | 749,000 | 2,100,000 | 75.0 | 2.8 |
| Western | 180,000 | 400,000 | 90.2 | 2.2 |
| Samoa | | | | |
| Micronesa | 108,000 | 150,000 | 132.9 | 1.4 |
| New | 161,000 | 100,000 | 182.0 | 0.6 |
| Caledonia | | | | |
| Hawaiians | 208,476 | 0 | 311.9 | 0.0 |

The results indicate a direct correlation between kava consumption and a corresponding reduction in cancer incidence. Age-standardized cancer incidence rates for kava drinking countries is one fourth to one third the cancer incidence found in non-kava drinking countries and non-kava drinking Polynesians. Other than Vanuatu, there is a direct correlation between kava consumption and reduced cancer incidence. The situation in Vanuatu is explained by a number of factors. The most obvious is that the chemopreventive effect of the detailed alpha-pyrones is dose related. It is understandable that after a certain amount of kava consumption no further chemoprotective benefit is derived. Another obvious factor is the number of people who do not drink kava. These individuals would be expected to have normal cancer rates and prevent the overall cancer incidence from dropping below a fixed level.

Normal cancer rates are found in the Pacific where kava is not consumed.

The data shows a strong inverse relationship between cancer incidence and kava consumption.

The results indicate a statistical correlation between kava consumption and a reduction in cancer incidence.

The data establishes that with kava consumption cancer incidence is reduced. The data establishes that the group of alpha-pyrones found in the kava plant are effective chemopreventive agents for cancer.

The alpha-pyrones known as kavapyrones may be extracted from the kava plant using one of a number of known extraction techniques. These compounds may also be synthesized according to a variety of processes described in the literature.

A physiologically accepted medium used to carry an effective amount of alpha-pyrone can be an inert carrier used in pill form, as a gum, in liquid form or a transdermal patch.

The alpha-pyrone compounds are preferably employed in doses ranging from approximately 5 mg to 600 mg every three to four hours, depending on the specific alpha-pyrone and the weight of the patient. Mode of administration: For the detailed alpha-pyrones to be effective as anticancer agents it is advised they be consumed on a regular basis. The described alpha-pyrones as chemoprotective agents for cancer can be supplied as a pill, gum, liquid or a transdermal patch.

What is claimed is:

1. A method of chemopreventing cancer by administering a cancer chemopreventive composition of matter, comprising an effective amount of at least one alpha-pyrone compound having the structural formula in which R1 is a hydrogen atom or an alkoxy radical having 1 to 4 carbon atoms, R2 is a hydrogen atom or a hydroxyl group, and R3 is an alkyl radical having from 1 to 4 carbon atoms or a styryl or phenethyl radical optionally substituted by one or two methylenedioxy radicals or one or two hydroxyl groups and/or one or two alkoxy radicals having from 1 to 4 carbon atoms, with the proviso that, when R2 is a hydroxyl group, then R3 is necessarily an unsubstituted phenethyl radical, with the proviso that when R3 is an alkyl radical having 1 to 4 carbon atoms, then R1 and R2 cannot both be hydrogen, in a physiologically acceptable carrier medium for the purpose of preventing cancer.

2. The method of claim 1, wherein said alpha-pyrone compound is one or more of the alpha-pyrones found in the plant Piper methysticum.

3. The method of claim 1, comprising a pill.

4. The method of claim 1, comprising a liquid.

5. The method of claim 1, comprising a gum.

6. The method of claim 1, comprising a transdermal patch.
